(19) United States
(12) Patent Application Publication
(10) Pub. No.: US 2023/0233441 A1
(43) Pub. Date: Jul. 27, 2023

(54) SKIN CARE COMPOSITION CONTAINING SODIUM HYALURONATE AND METHOD OF MAKING THE SAME

(71) Applicant: The Procter & Gamble Company, Cincinnati, OH (US)

(72) Inventors: Joseph Harry Jansen, Harrison, OH (US); Ellie Anne Martin, Lebanon, OH (US); Monica Salguero Marlow, Mason, OH (US); Cindy Lucille Lewis, West Chester Township, OH (US); George Frank Iaboni, JR., Springdale, OH (US); Renan Preda de Oliveira, West Chester, OH (US); Kathleen Elizabeth Riggins, Cincinnati, OH (US)

(21) Appl. No.: 17/582,484

(22) Filed: Jan. 24, 2022

(51) Int. Cl. A61K 8/73; A61K 8/06; A61K 8/81; A61K 8/891; A61K 8/67; A61Q 19/00

(52) U.S. Cl. CPC A61K 8/735; A61K 8/062; A61K 8/8147; A61K 8/8158; A61K 8/891; A61K 8/675; A61Q 19/00; A61K 2800/48; A61K 2800/87; A61K 2800/30

(57) ABSTRACT

A physically stable skin care composition with a continuous phase and a dispersed phase and a method for making the same. The continuous phase can include from about 0.5% to about 1.5% sodium hyaluronate with a weight average molecular weight from about 500,000 to about 1,000,000 Da according to the European Pharmacopoeia Method. The continuous phase can also include at least two thickening agents that can includes polyacrylamide polymers and copolymers and a carboxylic acid polymer. The dispersed phase can include silicone. The composition can have an average viscosity greater than 25,000 cP and the sodium hyaluronate can be fully hydrated.

### FIELD OF THE INVENTION

The present invention is generally directed towards skin care compositions that contain hyaluronic acid and one or more thickening agents, in particular a physically stable skin care emulsion composition that contains greater than 0.5% hyaluronic acid with a weight average molecular weight of from about 200,000 Da to about 1.5 million Da, two thickening agents, and an average viscosity greater than or equal to 25,000 cP (25 Pa*s).

### BACKGROUND OF THE INVENTION

Many consumers want skin care products that contain sodium hyaluronate, particularly a relatively high level of sodium hyaluronate (e.g., ≥0.5%, ≥0.7%, ≥0.9%, or ≥1%). Sodium hyaluronate can increase skin hydration and reduce the appearance of fine lines and wrinkles.

Some consumers want sodium hyaluronate in a cream/moisturizer intended for use on the face, neck, and/or eye area. These products are left on the skin, where the product can penetrate the skin to help repair, as well as remain on the surface to help protect skin from the outside elements.

Creams can be stored and dispensed from a jar. Many consumers like jars because they look great both when they are left out on a vanity or stored in a medicine cabinet, drawer, or shelf. It can also be satisfying for a consumer to scoop the cream out of a jar and apply it across the face, neck, and/or eye area. The jar's wide mouth also lets the user see the creamy, luscious texture of the product. Consumer acceptable creams stored in jars need to have sufficient viscosity, so they do not slosh out of the jar during handling and use.

However, it was found that adding a relatively high level of sodium hyaluronate to a cream, in particular a cream composition that contained traditional polymeric thickening agents, according to the typical method and order of addition for cream/moisturizers at production scale unexpectedly built an unmanageable level of viscosity. This resulted in a product where the sodium hyaluronate was not fully dispersed, which negated the hydration of sodium hyaluronate, and rendered the product unacceptable to consumers.

Therefore, there is a need for a cream/moisturizer that has sufficient viscosity so it can be stored in a jar and contains a relatively high level of sodium hyaluronate that is fully hydrated.

### SUMMARY OF THE INVENTION

A skin care composition comprising: (a) a continuous phase comprising: (i) from about 0.5% to about 1.5% sodium hyaluronate with a weight average molecular weight of from about 500,000 to about 1,000,000 Da, according to the European Pharmacopoeia Method; (ii) from about 0.1% to about 1% of a carboxylic acid polymer thickening agent; (iii) from about 0.8% to about 2.1% of polyacrylamide polymers and copolymer thickening agent; (iv) water; (b) a dispersed phase comprising silicone; wherein the sodium hyaluronate is fully hydrated; wherein the composition comprises a viscosity greater than 25,000 cP.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 shows the impact on viscosity of a simple system when adjusting the weight average molecular weight of 1% sodium hyaluronate;

FIG. 2 shows a photograph from a first wetting experiment with water and sodium hyaluronate;

FIG. 3 shows a photograph from a second wetting experiment with glycerin and sodium hyaluronate;

FIG. 4 shows the viscosity vs temperature for compositions containing glycerin, silicone(s), emulsifier, and/or sodium hyaluronate; and

FIG. 5 shows a schematic diagram of the production system.

### DETAILED DESCRIPTION OF THE INVENTION

Hyaluronic acid is a biological polymer that occurs naturally in the skin and is the natural mucopolysaccharide formed by bonding N-acetyl-O-glucosamine with glucuronic acid. Sodium hyaluronate is the sodium salt of hyaluronic acid and it can be used in skin care products including creams/moisturizers.

Many consumers want skin care compositions that contain sodium hyaluronate, because it can regulate water within skin's surface cells and keep skin hydrated throughout the day. The mechanism by which hyaluronic acid and salts thereof hydrate skin can also help prevent fine lines and wrinkles.

Many current marketed skin care compositions include relatively low levels of sodium hyaluronate. Some consumers may want skin care compositions that have a relatively high level (e.g., ≥0.5%, ≥0.7%, ≥0.9%, or ≥1%) of sodium hyaluronate, hoping to achieve even more moisture and/or anti-aging benefits. Many consumers want to apply skin care compositions with sodium hyaluronate in a cream, which generally contains thickening agents.

However, it can be difficult to formulate a homogenous emulsion where the sodium hyaluronate is fully hydrated. It was discovered that ˜1% sodium hyaluronate unexpectedly built viscosity in compositions, especially in compositions with one or more polymeric thickening agents. It was also surprisingly found that the amount of viscosity built was correlated with the molecular weight of the sodium hyaluronate.

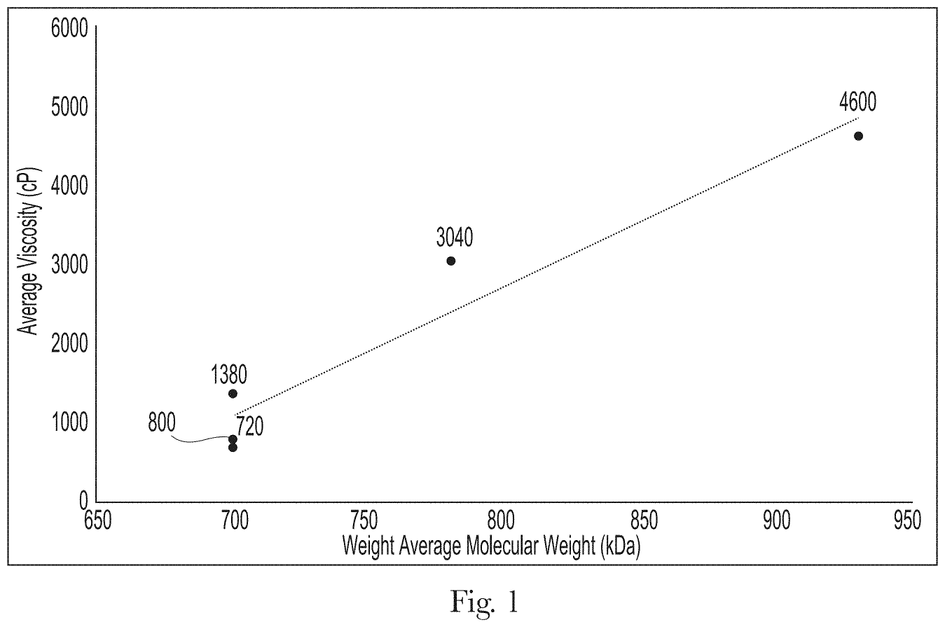

FIG. 1 shows the impact of the weight average molecular weight (Mw) of sodium hyaluronate in a simple system comprised of 7% Glycerin, 1% Sodium Hyaluronate, and the balance water on viscosity. FIG. 1 shows that as the Mw of the sodium hyaluronate increases, the viscosity increases.

The molecular weights were provided in a certificate of analysis by the supplier. The average viscosity was measured according to the Viscosity Test Method, described hereafter. The average viscosity was determined according to the following method: the viscosities for the simple system were measured after holding the sample for at least 24 hours at about 25° C. The viscosities of the examples are measured by a DV2T™-RV viscometer with RV3 spindle rotated at 5 rpm (available from Brookfield Engineering Laboratories, Middleboro, Massachusetts) at 25° C. The spindle is fully submerged, 2.5-3.0 cm below the composition's surface. The spindle is rotated for 30 s before starting data collection, then one data point is collected every 10 seconds for 5 readings, average (mean) the readings to determine the average viscosity.

Next, formulations, as described in Table 1, below, were made to evaluate the product stability and viscosity at different levels of thickening agent (Polyacrylamide & C13-14 Isoparaffin & Laureth-7 commercially available as Sepigel™ 305 from Seppic® Corporation and 0.45% Acrylates/C10-30 Alkyl Acrylate Crosspolymer) in combination with 1% sodium hyaluronate with different weight average molecular weights. The results are summarized in Table 2, below.

TABLE 1

| | Material | Example (wt. %) |
|---|---|---|
| Water Phase | Water | QS |
| | Sodium Hyaluronate¹ | 1 |
| | Polyacrylamide and | 0.9-1.8 |
| | isoparaffin and laureth-7² | (as indicated in Table 2) |
| | Acrylates/C10-30 Alkyl | 0.45 |
| | Acrylate Crosspolymer³ | |
| | Glycerin | 7 |
| | Niacinamide and other skin | 2.853 |
| | care actives | |
| | Chelant | 0.025 |
| | Antioxidant | 0.15 |
| | Preservative | 0.25 |
| | Emulsifer | 0.1 |
| Oil Phase | 5 cst Dimethicone | 4 |
| | Dimethicone and | 2 |
| | Dimethiconol⁴ | |
| | Emulsifiers | 0.4 |

¹The weight average molecular weight was 511,000 Da, 615,000 Da, 855,000 Da, or 920,000 Da, as indicated in Table 2, below.
²Sepigel ™ 305 from Seppic ® Corporation
³Carbopol ® Ultrez 21 from Lubrizol ®
⁴DC 1503 from Dow Coming ®

The average viscosity was determined ˜24 hours after the cream was made and after 2 weeks of storage at 60° C. If the average viscosity was less than or equal to 25,000 cP (25 Pa*s), as determined according to the Viscosity Test Method, after 2 weeks of storage at 60° C., it was not acceptable because it would be too thin to be easily used from a jar, as it would tend to slosh out during handling and use.

The weight average molecular weights of the sodium hyaluronate were provided in a certificate of analysis by the supplier.

TABLE 2

| | Mw of Sodium Hyaluronate | | | | | | | |
|---|---|---|---|---|---|---|---|---|
| | 511,000 Da | | 615,000 Da | | 855,000 Da | | 920,000 Da | |
| Sepigel ™ | Initial | 2 Weeks | Initial | 2 Weeks | Initial | 2 Weeks | Initial | 2 Weeks |
| 305 | Viscosity | at 60° C. | Viscosity | at 60° C. | Viscosity | at 60° C. | Viscosity | at 60° C. |
| (wt. %) | (cP) | (cP) | (cP) | (cP) | (cP) | (cP) | (cP) | (cP) |
| 0.9 | 24000 | 15000 | 32000 | 19000 | — | — | — | — |
| 1.2 | — | — | — | — | 53000 | 36000 | 58000 | 33000 |
| 1.5 | 33000 | 22000 | 44000 | 28000 | — | — | — | — |
| 1.8 | 38000 | 26000 | 52000 | 33000 | — | — | — | — |

Table 2 shows that the average product viscosity increases as the concentration of Sepigel™ 305 increased, as well as when the Mw of the sodium hyaluronate increased. The compositions containing sodium hyaluronate with a Mw of 511,000 Da and 0.9% and 1.5% Sepigel™ 305 and the composition containing sodium hyaluronate with a Mw of 615,000 Da at 0.9% Sepigel™ 305 did not have sufficient viscosity after two weeks at 60° C. and therefore are not consumer acceptable.

The Sepigel™ 305 dose response curves were not completed for the two higher Mw sodium hyaluronates (i.e., 855,000 Da and 920,000 Da), since the single point average viscosities were higher than that of the corresponding level of Sepigel™ 305 used with the lower Mw sodium hyaluronates, based upon the dose response curves. Therefore, it is expected that the higher MW sodium hyaluronates would have sufficient viscosity soon after they are made and after two weeks of storage at 60° C.

During benchtop production, it was observed that sodium hyaluronate had a greater impact on finished product viscosity than initially expected. To achieve full hydration of sodium hyaluronate when directly added to the water phase it was found that high shear energy and extended mix times were required. If not fully hydrated, gelation balls of sodium hyaluronate can be present in final product. The gelation balls can eventually hydrate, but the finished product may not be homogenous (i.e., the finished product can contain areas of high and low concentrations of sodium hyaluronate, which is not consumer acceptable). The challenge to fully hydrate and disperse sodium hyaluronate is exacerbated as the manufacturing scale is increased from benchtop to the production level.

FIG. 2 shows a photograph from a wetting experiment and shows what appears to be a bead of water 2 sitting on top of sodium hyaluronate powder 1. From this experiment, it was determined that sodium hyaluronate's rate of hydration can be instantaneous upon contact with water. Such instantaneous hydration can cause the sodium hyaluronate to bind and form a barrier around itself (i.e., form gelation balls of sodium hyaluronate). Typically, water diffuses into polymers; however, sodium hyaluronate's diffusion mechanism is reversed, sodium hyaluronate diffuse into water. It appears in FIG. 2 that a pure bead of water sits atop the bed of sodium hyaluronate powder, however, over time the sodium hyaluronate diffuses into the bead of water and the bead of water becomes gel like.

It was determined that if sodium hyaluronate were added directly to the water phase, like most actives, it would take significant time to reach full hydration (e.g., ˜4 to 24 hours or more for full scale productions, depending on production size). The problems with hydrating sodium hyaluronate to the water phase were exacerbated because the cream/moisturizer composition had two polymeric thickening agents (i.e., Sepigel™ 305 and Ultrez 21), which are needed to build sufficient viscosity in the composition, but also function via hydration/swelling in water, which further reduces the amount of free water in the system.

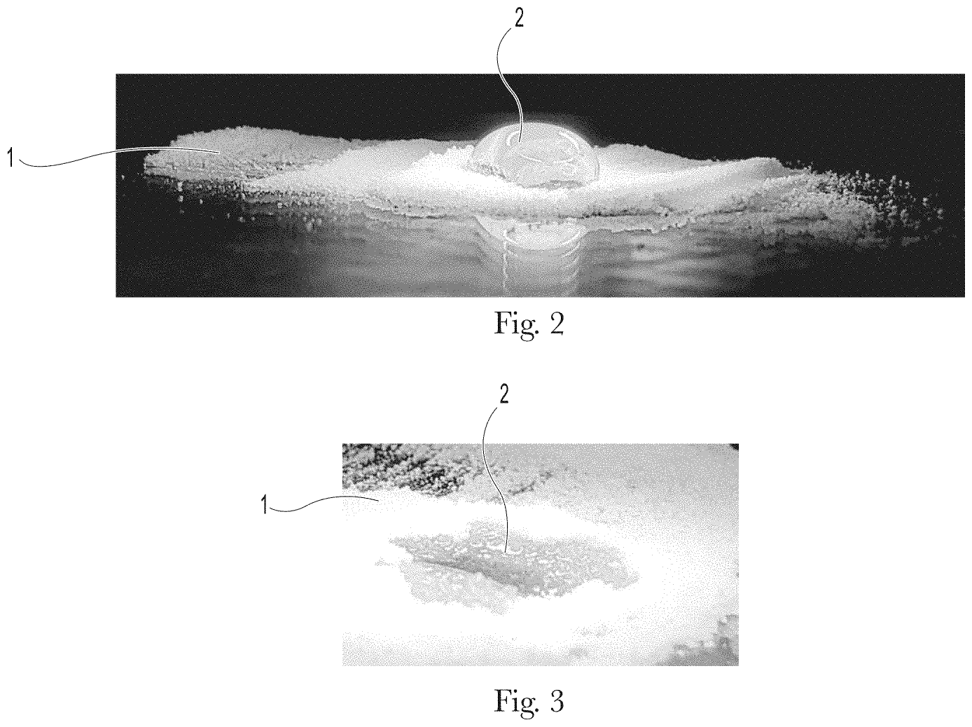

FIG. 3 shows a photograph from a second wetting experiment. FIG. 3 shows a pool of glycerin 3, which diffused into sodium hyaluronate powder 1. In this experiment, the sodium hyaluronate dispersed rapidly into the glycerin. It is believed that other oils, like silicone, would interact with sodium hyaluronate powder similarly to glycerin. Thus, it was determined that in order to reduce manufacturing time the sodium hyaluronate could be added to the water phase as part of a pre-mix, in particular a low water or water-free premix. The preferred premix could also have relatively low viscosity, in particular low viscosity without heating.

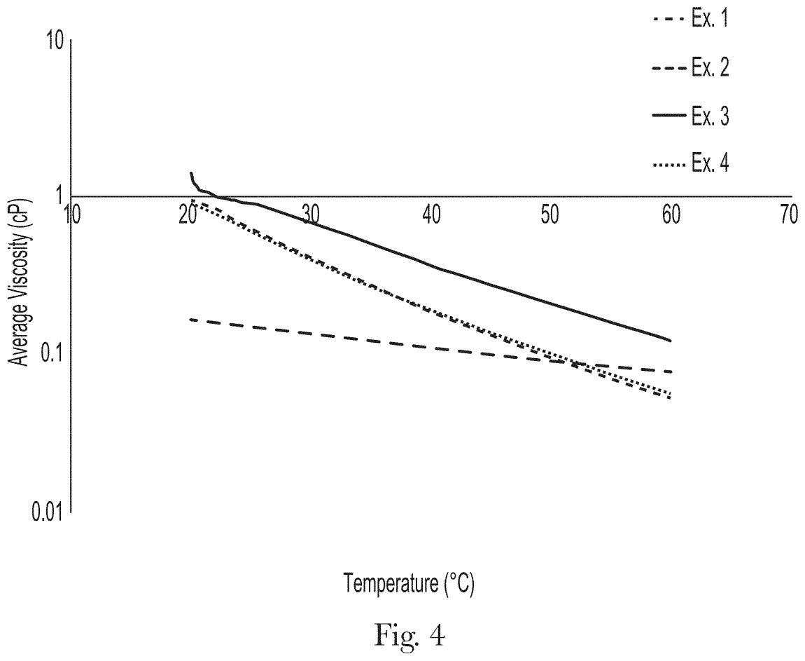

FIG. 4 shows the temperature vs. average viscosity for the Examples 1-4 in Table 3, below.

TABLE 3

| | Ex. 1 | Ex. 2 | Ex. 3 | Ex. 4 |
|---|---|---|---|---|
| | (wt. %) | (wt. %) | (wt. %) | (wt. %) |
| 5 cst Dimethicone | 22.99 | 45.45 | 18.35 | |
| Dimethicone and | | 45.45 | 18.35 | |
| Dimethiconol¹ | | | | |
| Polysorbate 20 | | 4.55 | 1.835 | |
| Laureth 4 | 2.3 | 4.55 | 1.835 | |
| Glycerin | 74.71 | | 59.63 | 100 |

¹DC1503 from Dow Corning ®

As shown in FIG. 4, the Example 2 curve generally had a lower average viscosity than glycerin between about 20° C. and 50° C., which indicated that silicone may be preferable for a premix over glycerin because it has a lower average viscosity without being heated, which reduces the time and energy consumption, which is especially important at large scale production. It is also interesting to note that Example 1, which included silicone and glycerin had an average viscosity dependance more like glycerin than silicone, and this may also be less preferred than a premix with predominantly silicone. Also, commercially available glycerin generally has a very small amount of water (˜1-2%) and since sodium hyaluronate hydrates instantly upon contact with water, it can be preferred to have a premix that is free of water (e.g., silicone or another oil).

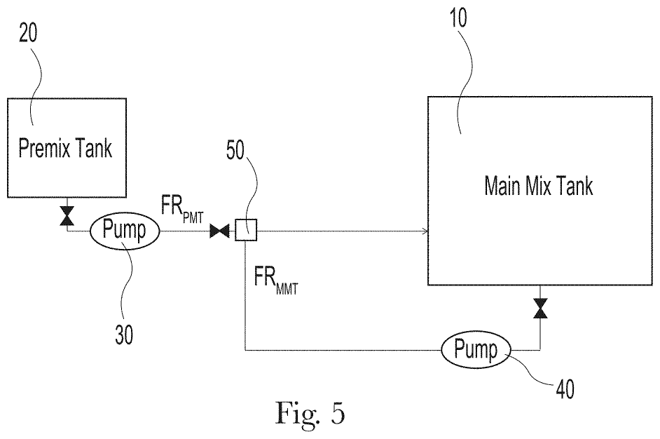

To make the cream/moisturizer compositions described herein, the following method can be used. First, a water phase is made with water soluble ingredients including, but not limited to, glycerin, chelant (e.g., disodium EDTA), water soluble skin care actives (e.g., panthenol), antioxidants (e.g., hydroxyacetophenone). In some examples, a thickening agent that does not swell until the pH of the composition is increased, like Carbopol® Ultrez 21, can be added to the water phase. FIG. 5 shows a schematic diagram of the production system. The water phase can be added to main mix tank 10 and stirred with a pitch blade turbine. The contents of main mix tank 10 can be recirculated by pump 40 to ensure bulk homogeneity. Water phase is mixed until the ingredients are in solution and/or dispersed. The water phase can contain about 70% to about 98%, alternatively from about 75% to about 95%, alternatively from about 80% to about 94%, alternatively from about 85% to about 93%, or alternatively from about 88% to about 92% water. Separately, a premix can be made. The premix can include the one or more silicones, sodium hyaluronate and emulsifiers. The premix can be mixed in a tank separate from the water phase, like premix tank 20. The sodium hyaluronate can be mixed until it is fully dispersed in the premix. The premix can be about 5% to about 35%, alternatively from about 7% to about 30%, alternatively from about 9% to about 25%, alternatively from about 11% to about 21%, or alternatively from about 13% to about 19% sodium hyaluronate.

The premix can be added to the water phase. Then, the mixture is milled to emulsify the silicone premix into the water phase, forming an emulsion where the water phase is the continuous phase, and the silicone droplets are dispersed in the internal phase. Since sodium hyaluronate is hydrophilic and swellable in water, the dispersed sodium hyaluronate leaves the oil phase during milling/recirculation and comes to equilibrium in the water phase where it can be fully hydrated in water.

In examples where the premix is added to the main mix in line at transfer injection port 50, as shown in FIG. 5, it was surprisingly found that if the premix, which contains sodium hyaluronate, was blended into the water phase too quickly, high pressure could build in the system, since the full surface area of the hyaluronic acid was exposed and instantly hydrates when it becomes exposed to water. When the pressure in the system is greater than the system pressure rating, the pressure valve releases, and the system stops recirculating. It was determined that the ratio of hyaluronic acid to water can be important to prevent the pressure from building up too quickly in the system. When the premix and the main mix first meet in line, there is less than 3%, alternatively less than 2.5%, alternatively less than 2.9%, alternatively less than 2.5%, alternatively less than 2.25%, alternatively less than 2.0%, alternatively less than 1.9%, alternatively less than 1.8%, alternatively less than 1.7% sodium hyaluronate at the transfer injection port. As the sodium hyaluronate is added to the system, the level of sodium hyaluronate at the transfer injection port increases above the level it when the premix was first added. When all the premix is added there can be greater than 1.75%, alternatively greater than 2%, alternatively greater than 2.2%, alternatively greater than 2.4%, alternatively greater than 2.6%, alternatively greater than 2.8%, alternatively greater than 2.85%, alternatively greater than or equal to 2.9%, alternatively greater than or equal to 2.95% sodium hyaluronate at the transfer injection port.

After the emulsion is formed, the additional ingredients can be added. In some examples, niacinamide can be added. If added, niacinamide can change the pH and can cause pH sensitive thickening agents (e.g., Carbopol© Ultrez 21), if present, to swell. Other thickening agents (e.g., Sepigel™ 305), preservatives (e.g., phenoxyethanol), additional skin care actives (e.g., promatrixyl), pH adjusters (e.g., aminomethyl propanol), emollients (e.g., ethylhexylglycerin), and optional ingredients can also be added.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

### Definitions

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein. In a specific example, an effective amount of a vitamin B₃ compound is an amount sufficient to improve the health and/or appearance of psoriatic skin during a treatment period. In some instances, an effective amount may be demonstrated using ex vivo and/or in vitro methods.

“Improve the appearance of” means providing a measurable, desirable change or benefit in skin appearance, which may be quantified, for example, by a decrease in redness, inflammation, and/or plaque scales.

“Safe and effective amount” means an effective amount of an ingredient that is low enough to avoid serious side effects (within the scope of sound medical judgment).

“Skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Treatment period” means the length of time and/or frequency that a material or composition is applied to a target skin surface.

### Composition

The skin care composition can be a moisturizer/cream with a viscosity that is sufficient for it to be dispensed from a jar without sloshing and is also thin enough so it can be easily spread across a user's face, eye area, and/or neck. The average viscosity can be greater than 25,000 cP (25 Pa*s), alternatively greater than or equal to 27,000 cP (27 Pa*s), or alternatively greater than or equal to 30,000 cP (30 Pa*s). The average viscosity can be from about 23,000 cP (23 Pa*s) to about 75,000 cP (75 Pa*s), alternatively from about 24,000 cP (24 Pa*s) to about 68,000 cP (68 Pa*s), or alternatively from about 25,000 cP (25 Pa*s) to about 60,000 cP (60 Pa*s). The average viscosity can be determined by the Viscosity Test Method, described hereafter.

The composition can contain greater than or equal to 0.5%, alternatively greater than or equal to 0.6%, alternatively greater than or equal to 0.7%, alternatively greater than or equal to 0.8%, alternatively greater than or equal to 0.9%, or alternatively greater than or equal to 1.0% sodium hyaluronate. The composition can contain from about 0.5% to about 5%, alternatively from about 0.6% to about 4%, alternatively from about 0.75% to about 3%, alternatively from about 0.9% to about 2%, alternatively from about 1% to about 1.5% sodium hyaluronate.

The sodium hyaluronate can have a Mw of from about 200,000 Da to about 1.5 million Da, alternatively from about 300,000 Da to about 1.25 million Da, alternatively from about 400,000 Da to about 1 million Da, or alternatively from about 500,000 Da to about 950,000 Da. The sodium hyaluronate can have a Mw of less than 3 million Da, alternatively less than 2 million Da, alternatively less than 1 million Da. The sodium hyaluronate can have a Mw of greater than 100,000 Da, alternatively greater than 200,000 Da, alternatively greater than 300,000 Da, alternatively greater than 400,000 Da, alternatively greater than about 500,000 Da. The Mw of sodium hyaluronate can be determined according to the method described in the European Pharmacopoeia (European Pharmacopoeia 9.0. Sodium Hyaluronate. 01/2017).

The skin care composition can contain about 1% sodium hyaluronate with a Mw of from about 300,000 Da to about 1 million Da, alternatively from about 400,000 Da to about 950,000 Da, alternatively from about 500,000 Da to about 920,000 Da, alternatively from about 505,000 Da to about 855,000 Da, or alternatively from about 510,000 Da to about 620,000 Da and greater than 1.6% thickening agent, alternatively greater than 1.7% thickening agent, alternatively greater than 1.75% thickening agent, or alternatively greater than or equal to about 1.8% thickening agent.

The skin care composition can contain about 1% sodium hyaluronate with a Mw of from about 500,000 Da to about 1 million Da, alternatively from about 550,000 Da to about 950,000 Da, alternatively from about 575,000 Da to about 920,000 Da, alternatively from about 600,000 Da to about 855,000 Da, alternatively from about 610,000 Da to about 800,000, alternatively from about 615,000 Da to about 750,000 Da, or alternatively from about 615,000 Da to about 700,000 Da and greater than 1.3% thickening agent, alternatively greater than 1.4% thickening agent, or alternatively greater than or equal to 1.5% thickening agent.

The skin care composition can contain about 1% sodium hyaluronate with a Mw of from about 700,000 Da to about 2 million Da, alternatively from about 750,000 Da to about 1.5 million Da, alternatively from about 800,000 Da to about 1.25 million Da, alternatively from about 825,000 Da to about 1 million Da, alternatively from about 850,000 Da to about 950,000 Da, or alternatively from about 855,000 Da to about 920,000 Da and greater than 0.5% thickening agent, alternatively greater than 0.75% thickening agent, greater than 1% thickening agent, greater than 1.1% thickening agent, or alternatively greater than or equal to 1.2% thickening agent.

The skin care composition can be phthalate free, paraben free, dye-free, free of synthetic fragrance, and/or fragrance free.

### Thickening Agent

The composition may include one or more thickening agents, alternatively at least two thickening agents. The compositions may comprise greater than 1%, alternatively greater than 1.2%, alternatively greater than 1.4%, alternatively greater than 1.5%, alternatively greater than 1.55%, alternatively greater than 1.6%, or alternatively greater than or equal to 1.65% thickening agent. The compositions may comprise less than or equal to 2.5%, alternatively less than or equal to 2.4%, alternatively less than or equal to 2.3%, or alternatively less than or equal to 2.25% thickening agent. The compositions may comprise from about 1% to about 3%, alternatively from about 1.2% to about 2.75%, alternatively from about 1.5% to about 2.5%, or alternatively from about 1.6% to about 2.3% of a thickening agent. Suitable classes of thickening agents include but are not limited to carboxylic acid polymers, polyacrylamide polymers, sulfonated polymers, copolymers thereof, hydrophobically modified derivatives thereof, and mixtures thereof.

The composition can include a carboxylic acid polymer thickening agent such as a carbomer. The composition can contain from about 0.1% to about 1%, alternatively from about 0.2% to about 0.8%, alternatively 0.25% to about 0.7%, alternatively from about 0.3% to about 0.6%, and alternatively from about 0.4% to about 0.5% of a carboxylic acid polymer thickening agent.

The composition can include a polyacrylamide polymer and copolymer thickening agent. The composition can contain from about 0.8% to about 2.1%, alternatively from about 0.9% to about 2%, alternatively from about 1% to about 1.9%, alternatively from about 1.1% to about 1.8%, alternatively from about 1.2% to about 1.7%, or alternatively from about 1.3% to about 1.6% polyacrylamide polymer and copolymer thickening agent.

Suitable thickening agents include carboxylic acid polymers such as the carbomers (e.g., the CARBOPOL® 900 series such as CARBOPOL® 954), and Ultrez 10 and Ultrez 30. Other suitable carboxylic acid polymeric agents include copolymers of C₁₀₋₃₀ alkyl acrylates with one or more monomers of acrylic acid, methacrylic acid, or one of their short chain (i.e., C₁₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, Ultrez 20, Ultrez 21, PEMULEN TR-1, and PEMULEN TR-2, from Noveon, Inc.

Other suitable thickening agents include the polyacrylamide polymers and copolymers. An exemplary polyacrylamide polymer has the CTFA designation “polyacrylamide and isoparaffin and laureth-7” and is available under the trade name SEPIGEL 305 from Seppic® Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson, N.J.).

Other suitable thickening agents useful herein are sulfonated polymers such as the CTFA designated sodium polyacryloyldimethyl taurate available under the trade name Simulgel 800 from Seppic® Corp. and Viscolam® at 100 P available from Lamberti S.p.A. (Gallarate, Italy). Another commercially available material comprising a sulfonated polymer is Sepiplus™ 400 available from Seppic® Corp.

Further, suitable thickening agents may include superabsorbent polymers. These superabsorbent polymers may be chosen from: crosslinked sodium polyacrylates, such as, for example, those sold under the names Octacare X100, X110 and RM100 by Avecia®, those sold under the names Flocare GB300 and Flosorb 500 by SNF™, those sold under the names Luquasorb 1003, Luquasorb 1010, Luquasorb 1280 and Luquasorb 1100 by BASF@, those sold under the names Water Lock G400 and G430 (INCI name: Acrylamide/Sodium Acrylate Copolymer) by Grain Processing@, or Aqua Keep® 10 SH NF, Aqua Keep® 10 SH NFC, sodium acrylate crosspolymer-2, provided by Sumitomo Seika, starches grafted by an acrylic polymer (homopolymer or copolymer) and in particular by sodium polyacrylate, such as those sold under the names Sanfresh ST-100C, ST100MC and IM-300MC by Sanyo Chemical Industries@, Makimousse 12 and Makimouse 25 supplied by Kobo Products Inc (INCI name: Sodium Polyacrylate Starch), hydrolysed starches grafted by an acrylic polymer (homopolymer or copolymer), in particular the acryloacrylamide/sodium acrylate copolymer, such as those sold under the names Water Lock A-240, A-180, B-204, D-223, A-100, C-200 and D-223 by Grain Processing@ (INCI name: Starch/Acrylamide/Sodium Acrylate Copolymer). Preferred superabsorbent polymers can include Makimousse 12 and Makimousse 25.

Suitable thickening agents for use herein include gums. “Gum” is a broadly defined term in the art. Gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, derivatives thereof and mixtures thereof.

Natural gums are polysaccharides of natural origin, capable of causing a large viscosity increase in solution, even at small concentrations. They can be used as thickening agents, gelling agents, emulsifying agents, and stabilizers. Most often these gums are found in the woody elements of plants or in seed coatings. Natural gums can be classified according to their origin. They can also be classified as uncharged or ionic polymers (polyelectrolytes), examples of which include the following. Natural gums obtained from seaweeds, such as: agar; alginic acid; sodium alginate; and carrageenan. Natural gums obtained from non-marine botanical resources include: gum arabic, from the sap of Acacia trees; gum ghatti, from the sap of Anogeissus trees; gum tragacanth, from the sap of Astragalus shrubs; karaya gum, from the sap of Sterculia trees. Examples of uncharged gums include: guar gum, from guar beans, locust bean gum, from the seeds of the carob tree; beta-glucan, from oat or barley bran; chicle gum, an older base for chewing gum obtained from the chicle tree; dammar gum, from the sap of Dipterocarpaceae trees; glucomannan from the konjac plant; mastic gum, a chewing gum from ancient Greece obtained from the mastic tree; psyllium seed husks, from the Plantago plant; spruce gum, a chewing gum of American Indians obtained from spruce trees; tara gum, from the seeds of the tara tree. Natural gums produced by bacterial fermentation include gellan gum and xanthan gum.

Vitamin B₃ compound

The compositions herein can include a safe and effective amount of a vitamin B₃ compound. In some instances, the present compositions may contain 0.01% to 10%, by weight, of the vitamin B₃ compound, based on the weight or volume of the composition (e.g., 0.1% to 10%, 0.5% to 5%, or even 1% to 3%).

As used herein, “vitamin B₃ compound” means a compound having the formula:

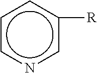

Where: R is CONH₂ (i.e., niacinamide), COOH (i.e., nicotinic acid) or CH₂OH (i.e., nicotinyl alcohol); derivatives thereof; and salts of any of the foregoing.

Exemplary derivatives of vitamin B3 compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion that has a continuous aqueous phase (e.g., an oil-in-water or water-in-oil-in-water emulsion) or a continuous oil phase (e.g., water-in-oil or oil-in-water-in-oil emulsion). The oil phase of the emulsion may include silicone oils, non-silicone oils such as hydrocarbon oils, esters, ethers, and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). The composition can contain from about 1% to about 15%, alternatively from about 3% to about 10%, alternatively from about 4% to about 9%, and alternatively from about 5% to about 8% humectant.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., Mw of 425 to 2025 g/mole), glycerol, butylene glycol, 1,2,4-butanetriol, sorbitol esters, 1,2,6-iexanetriol, ethanol, isoprop anol, sorbitol esters, butanediol, ether propanol, ethoxylated ethers, propoxylated ethers and combinations thereof.

### Silicone Oil

The composition can include a silicone oil selected from volatile silicone oil, non-volatile silicone oil, and combinations thereof. The silicone oil can be in the dispersed phase. The composition can include from about 1% to about 10% silicone oil, alternatively from about 3% to about 8% silicone oil, and alternatively from about 4% to about 7% silicone oil.

### Volatile Silicone Oil

Suitable volatile silicones include cyclic and linear volatile silicones. A description of various volatile silicones is found in Todd, et al. “Volatile Silicone Fluids for Cosmetics”, 91 Cosmetics and Toiletries 27-32 (1976). Suitable cyclic volatile silicones include cyclic dimethyl siloxane chains containing an average of from about 3 to about 5 silicon atoms, preferably from about 4 to about 5 silicon atoms. Exemplary cyclic volatile silicones of varying viscosities include Dow Corning DC 244, DC 245, DC 344, and DC 345; GE Silicones-OSi Specialties Volatile Silicone 7207 and Volatile Silicone 7158; and GE Silicones SF1202. Suitable volatile linear silicones include the polydimethylsiloxanes containing an average of from about 2 to about 8 silicon atoms. Exemplary linear volatile silicones include the Dow Corning DC 200 series with viscosities of 0.65 est, 1.0 cst, and 2.0 cst. In certain embodiments, the linear volatile silicones generally have viscosities of less than or equal to about 4 centistokes at 25° C., and the cyclic materials generally have viscosities of less than about 6 centistokes at 25° C.

### Non-Volatile Silicone Oils

Suitable non-volatile silicone oils include polysiloxanes. Non-volatile polylsiloxanes may have a viscosity of from about 10 to about 1,000,000 centistokes at 25° C. Such polysiloxanes can be represented by the general chemical formula:

R3SiO[R2SiO]×SiR3

wherein each R is independently selected from hydrogen or C1-30 straight or branched chain, saturated or unsaturated alkyl, phenyl or aryl, trialkylsiloxy; and x is an integer from 0 to about 10,000. In certain embodiments, R is methyl or ethyl. Commercially available polysiloxanes include the polydimethylsiloxanes, which are also known as dimethicones, examples of which include the DM-Fluid series from Shin-Etsu, the Vicasil® series sold by Momentive Performance Materials Inc., and the Dow Corning® 200 series sold by Dow Corning Corporation. Specific examples of suitable polydimethylsiloxanes include Dow Corning® 200 fluids (also sold as Xiameter® PMX-200 Silicone Fluids). Suitable dimethicones include those represented by the chemical formula:

R3SiO[R2SiO]×[RR′SiO]ySiR3

wherein R and R′ are each independently hydrogen or C1-30 straight or branched chain, saturated or unsaturated alkyl, aryl, or trialkylsiloxy; and x and y are each integers of 1 to 1,000,000. Examples include alkyl dimethicones wherein at least R′ is a fatty alkyl (e.g., C12-22). A suitable alkyl dimethicone is cetyl dimethicone, wherein R′ is a straight C16 chain and R is methyl, commercially available as 2502Cosmetic Fluid from Dow Corning.

Preferred non-volatile oils include dimethicones (polydimethylsiloxanes), preferably with viscosities of between 10 cst and 1000 cst, more preferably between 15 cst to 400 cst, most preferably between 20 cst and 200 cst. The average chain lengths for these preferred dimethicone materials is from about 12 to about 375 dimethylsiloxane units, more preferably from about 20 to about 200 dimethylsiloxane units, and most preferably with average chain lengths of from about 27 to about 125 dimethylsiloxane units. In one embodiment, the second composition will comprise at least one non-volatile silicone oil. In one such embodiment, at least about 70%, by weight of the non-volatile oil, is a non-volatile silicone oil. In another embodiment, at least about 80%, by weight of the nonvolatile oil, is a non-volatile silicone. In yet another embodiment, at least about 90%, by weight of the non-volatile oil, is a non-volatile silicone oil.

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. In some examples, the composition can include a fragrance, in particular a natural fragrance, or a colorant, in particular a natural colorant. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Method of Use

The skin care cream/moisturizer composition may be applied to the face, neck, and/or a portion or combination thereof at least once a day, twice a day, or on a more frequent daily basis, during a treatment period. When applied twice daily, the first and second applications are separated by at least 1 to 12 hours. Typically, the composition is applied in the morning and/or at night before bed. The treatment period herein is ideally of sufficient time for the sodium hyaluronate and/or other skin care actives to improve the appearance of the skin. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The cream can be intended for use before bedtime and/or in the morning. It can be evenly massaged over the entire face (including or excluding the eye area) and/or neck. The product can be the final step in a skin care routine and can be applied after cleansing and optionally after applying serums and/or other skin care products.

The cream can be intended to help improve the appearance of fine lines and wrinkles around a user's eyes. The cream can be used in the morning and/or at night and can be gently massaged until thoroughly absorbed into the eye area—under eyes, outer corners, and/or eyelids.

The cream can be stronger and last longer, up to two times longer than other commercially available products. The cream can leave skin dewy and hydrated.

### Examples

The example in Table 4, can be prepared according to the method described herein.

| | Ex. A | Ex. B | Ex. C | Ex. D | Ex. E | Ex. F | Ex. G |
|---|---|---|---|---|---|---|---|
| Water Phase | | | | | | | |
| Water | QS | QS | QS | QS | QS | QS | QS |
| Dex-Panthenol | 0.5 | 0.5 | 0.5 | — | — | — | 0.5 |
| Glycerin | 7 | 10 | — | 3 | — | 7 | 15 |
| Butylene Glycol | — | — | 4 | — | — | — | — |
| Disodium EDTA | 0.05 | 0.025 | 0.05 | 0.025 | 0.025 | — | 0.05 |
| Hydroxyacetophenone | 0.15 | 0.15 | 0.05 | — | 0.15 | — | 0.2 |
| Ethylhexylglycerine | 0.2 | 0.1 | — | — | — | — | — |
| Phenoxyethanol | 0.05 | 0.25 | 0.5 | — | 0.15 | — | 0.2 |
| Symdiol 68¹ | — | — | — | 0.4 | — | — | — |
| Glycacil L² | — | — | — | 0.09 | — | — | — |
| Glydant Plus Liquid³ | — | — | — | — | — | 0.3 | — |
| Niacinamide | 2.0 | 2.0 | 5.0 | 3.5 | — | 2.0 | 3.0 |
| Sepiwhite MSH⁴ | — | — | — | — | 1.0 | — | — |
| Glyco-Repair⁵ | — | — | 2.0 | — | 1.0 | — | — |
| Palestrina⁶ | 0.6 | — | 0.4 | — | 1.0 | — | — |
| Olivem 460⁷ | 0.1 | — | — | 0.1 | — | — | — |
| Promatrixyl⁸ | 0.4 | — | 0.7 | 0.05 | — | — | 0.3 |
| Green Tea Extract | — | — | — | — | — | 1.0 | 1.0 |
| pH Adjustor | | | | | | | |
| Triethanolamine | 0.45 | — | 0.1 | — | — | — | — |
| Aminomethyl | — | 0.35 | — | 0.1 | — | 0.1 | — |
| Propanol | | | | | | | |
| Thickener | | | | | | | |
| Sepigel 305⁹ | 0.9 | 1.5 | 2.0 | — | 0.9 | 1.5 | — |
| Simulgel INS-100¹⁰ | — | — | — | 0.9 | — | — | 2.0 |
| Makimousse-12¹¹ | — | — | — | — | 0.5 | — | 0.5 |
| Ultrez-10¹² | 0.2 | — | 0.1 | 0.1 | — | 0.1 | — |
| Ultrez-21¹³ | — | 0.45 | — | 0.1 | — | 0.1 | — |
| Xanthan Gum | — | — | — | — | — | 0.1 | 0.1 |
| Oil Phase | | | | | | | |
| Sodium Hyaluronate | 0.5 | 1.5 | 1.0 | 0.75 | 1.0 | 1.0 | 0.5 |
| Cyclomethicone D5 | 4.0 | 1.0 | 2.0 | — | — | — | — |
| Dimethicone 2 cst | 2.0 | — | 2.0 | — | — | — | — |
| Dimethicone 5 cst | | 5.0 | | 3.0 | | 4.0 | 2.0 |
| Dimethicone 50 cst | | | | 2.0 | 3.0 | 2.0 | — |
| Dimethicone 350 cst | — | — | 1.0 | — | — | — | — |
| DC9041¹⁴ | 0.5 | — | — | 1.0 | — | — | 2.0 |
| Hexyldecanol | — | — | — | 0.1 | 5.0 | — | — |
| Retinyl Propionate | 0.3 | — | — | 0.3 | — | — | — |
| Laureth-4 | 0.1 | 0.2 | 0.3 | — | 0.2 | 0.1 | 0.3 |
| DC1503¹⁵ | — | 2.0 | — | — | 1.5 | — | — |
| Polysorbate 20 | 0.1 | 0.2 | — | 0.3 | 0.2 | 0.2 | — |

¹1,2-hexanediol and caprylyl glycol, from Symrise ®
²Iodopropynyl butylcarbamate, PEG-4 laurate, PEG-4 dilaurate, and polyethylene glycol, from Lonza ®
³DMDM Hydantoin, Butane-1,3-diol, iodopropynyl butylcarbamate, water, from Lonza ®
⁴Undecylenoyl phenylalanine, from Seppic ® Corporation
⁵Water and hydrolyzed ceratonia siliqua seed extract, from Silab ®
⁶Water, glycerin, decyl glucoside, lactic acid, benzyl alcohol, and palmitoyl dipeptide-7, from Sederma ®
⁷Sodium PEG-7 olive oil carboxylate, from B&T S.r.l.
⁸Water, glycerin, PEG-100 stearate, benzyl alcohol, and palmitoyl pentapeptide-4, from Sederma
⁹Polyacrylamide, C13-14 Isoparaffin, and laureth-7, from Seppic ® Corporation
¹⁰Sodium polyacrylate starch, from Kobo ® Products Inc.
¹²Carbomer, from Lubrizol ®
¹³Acrylates C10-30 alkyl acrylate crosspolymer, from Lubrizol ®
¹⁴Dimethicone (and) dimethicone crosspolymer, from Dow Corning ®
¹⁵Dimethicone (and) dimethiconol, from Dow Corning ®

### TEST METHODS

### Viscosity Test Method

After storing for at least 24 hours at 25° C., the product viscosity is measured by a DV2T-RV viscometer (available from Brookfield Engineering Laboratories, Middleboro, Massachusetts) with a TC spindle rotated at 5 rpm at 25° C. The spindle starts 2.5-3.0 cm below the product surface and travels a helipath downward and collects one reading every 9 seconds for 5 readings. The average (mean) of the readings is calculated to determine the average viscosity.

### Combinations

A. A skin care composition comprising:
a. a continuous phase comprising:
   i. from about 0.5% to about 1.5% sodium hyaluronate with a weight average molecular weight of from about 500,000 to about 1,000,000 Da, according to the European Pharmacopoeia Method;
   ii. from about 0.1% to about 1% of a carboxylic acid polymer thickening agent;
   iii. from about 0.8% to about 2.1% of polyacrylamide polymers and copolymer thickening agent;
   iv. water;
b. a dispersed phase comprising silicone;
   wherein the sodium hyaluronate is fully hydrated;
   wherein the composition comprises an average viscosity greater than 25,000 cP, according to the Viscosity Test Method.

B. The composition according to Paragraph A, wherein the composition is physically stable.

C. The composition according to Paragraph B, wherein the composition is homogenous.

D. The composition according to Paragraphs A-C, wherein the weight average molecular weight of the sodium hyaluronate is from about 500,000 Da to about 920,000 Da, more preferably from about 505,000 Da to about 855,000 Da, and even more preferably from about 510,000 Da to about 620,000, according to the European Pharmacopoeia Method.

E. The composition according to Paragraphs A-D, wherein the weight average molecular weight of the sodium hyaluronate is from about 700,000 Da to about 1,000,000 Da, preferably from about 750,000 Da to about 1,000,000 Da, more preferably from about 800,000 Da to about 1,000,000 Da, and even more preferably from about 855,000 Da to about 920,000 Da, according to the European Pharmacopoeia Method.

F. The composition according to Paragraphs A-E, wherein the average viscosity is greater than or equal to 27,000 cP (27 Pa*s), and preferably greater than or equal to 30,000 cP (30 Pa*s), according to the Viscosity Test Method, herein.

G. The composition according to Paragraphs A-E, wherein the average viscosity is from about 25,000 cP (25 Pa*s) to about 75,000 cP (75 Pa*s), preferably from about 25,000 cP (25 Pa*s) to about 68,000 cP (68 Pa*s), and more preferably from about 25,000 cP (25 Pa*s) to about 60,000 cP (60 Pa*s) according to the Viscosity Test Method, herein.

H. The composition according to Paragraphs A-G, wherein the composition is free of phthalate, paraben, dye, synthetic fragrance, and/or fragrance.

I. The composition according to Paragraphs A-H, wherein the water phase further a vitamin B₃ compound selected from niacinamide, nicotinic acid, nicotinyl alcohol, and combinations thereof

J. The composition according to Paragraphs A-I, wherein the continuous phase further comprises from about 3% to about 10% glycerin, preferably from about 4% to about 9% glycerin, and more preferably from about 5% to about 8% glycerin.

K. The composition according to Paragraphs A-J, wherein the polyacrylamide polymers and copolymer thickening agent comprises Polyacrylamide & C13-14 Isoparaffin & Laureth-7.

L. The composition according to Paragraphs A-K, wherein the carboxylic acid polymer thickening agent comprises Acrylates/C10-30 Alkyl Acrylate Crosspolymer.

M. The composition according to Paragraphs A-L, wherein the composition comprises from about 0.2% to about 0.8% of a carboxylic acid polymer thickening agent, preferably from about 0.25% to about 0.7% of a carboxylic acid polymer thickening agent, more preferably from about 0.3% to about 0.6% of a carboxylic acid polymer thickening agent, and most preferably from about 0.4% to about 0.5% of a carboxylic acid polymer thickening agent.

N. T The composition according to Paragraphs A-N, wherein the composition comprises from about 0.9% to about 2% polyacrylamide polymer and copolymer thickening agent, preferably from about 1% to about 1.9% polyacrylamide polymer and copolymer thickening agent, more preferably from about 1.1% to about 1.8% polyacrylamide polymer and copolymer thickening agent, even more preferably from about 1.2% to about 1.7% polyacrylamide polymer and copolymer thickening agent, and most preferably from about 1.3% to about 1.6% polyacrylamide polymer and copolymer thickening agent.

O. A skin care product comprising a jar adapted for storing the skin care composition according to Paragraphs A-N.

P. Use of the skin care serum composition of Paragraphs A-N to increase skin hydration and/or reduce the appearance of fine lines or wrinkles.

Q. A method for making the skin care composition according to Paragraphs A-N comprising:
a. preparing a homogenous main mix comprising a carboxylic acid polymer thickening agent and from about 70% to about 98% water, preferably from about 75% to about 95%, more preferably from about 80% to about 94%, and most preferably from about 85% to about 93%;
b. preparing a premix comprising silicone and from about 5% to about 35% sodium hyaluronate, preferably from about 7% to about 30%, more preferably from about 9% to about 25%, even more preferably from about 11% to about 21%, and most preferably from about 13% to about 19% sodium hyaluronate;
c. adding the premix to the main mix and milling the mixture to emulsify the silicone premix into the main mix to form an emulsion;
d. adding a polyacrylamide polymer and copolymer thickening agent to the emulsion to form a fully hydrated, physically stable skin care composition comprising a viscosity greater than 25,000 cP.

R. The method according to Paragraph Q, wherein the method is performed without adding heat.

S. The method according to Paragraphs Q-R, wherein the main mix further comprises glycerin.

T. The method according to Paragraphs Q-S, wherein the premix is substantially free of water, preferably free of water.

U. The method according to Paragraphs Q-T, wherein the physically stable skin care composition is homogenous.

V. The method according to Paragraphs Q-U, wherein the premix is added to the main mix in line at a transfer injection port.

W. The method according to Paragraphs Q-V, wherein the amount of sodium hyaluronate in line at the transfer injection port when the premix and main mix first meet is less than 2% sodium hyaluronate, preferably less than 1.9% sodium hyaluronate, more preferably less than 1.8% sodium hyaluronate, and most preferably less than 1.7% sodium hyaluronate.

X. The method according to Paragraphs Q-W, wherein one or more additional ingredients are added to the emulsion; wherein the additional ingredients selected from vitamin B₃ compound, preservative, additional skin care active, pH adjuster, emollient, and combinations thereof.

Y. The method according to Paragraphs Q-X, wherein the additional ingredient comprises a vitamin B₃ compound selected from niacinamide, nicotinic acid, nicotinyl alcohol, and combinations thereof is added to the emulsion.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A skin care composition comprising:
a. a continuous phase comprising:
i. from about 0.5% to about 1.5% sodium hyaluronate with a weight average molecular weight of from about 500,000 to about 1,000,000 Da;
ii. from about 0.1% to about 1% of a carboxylic acid polymer thickening agent;
iii. from about 0.8% to about 2.1% of a polyacrylamide polymer and copolymer thickening agent;
iv. water
b. a dispersed phase comprising silicone;
wherein the sodium hyaluronate is fully hydrated;
wherein the composition comprises an average viscosity greater than 25,000 cP.

2. The composition of claim 1, wherein the composition is physically stable.

3. The composition of claim 1, wherein the composition is homogenous.

4. The composition of claim 1, wherein the composition is free of phthalate, paraben, dye, synthetic fragrance, and/or fragrance.

5. The composition of claim 1, wherein the sodium hyaluronate comprises a weight average molecular weight from about 700,000 to about 1,000,000 Da.

6. The composition of claim 1, wherein the water phase further a vitamin B₃ compound selected from niacinamide, nicotinic acid, nicotinyl alcohol, and combinations thereof

7. The composition of claim 1, wherein the continuous further comprises from about 3% to about 10% glycerin.

8. The composition of claim 1, wherein the polyacrylamide polymer and copolymer thickening agent comprises Polyacrylamide & C13-14 Isoparaffin & Laureth-7.

9. The composition of claim 1, wherein the carboxylic acid polymer thickening agent comprises Acrylates/C10-30 Alkyl Acrylate Crosspolymer.

10. A skin care product comprising a jar adapted for storing the skin care composition of claim 1.

11. A method for making a physically stable skin care composition comprising:
a. preparing a homogenous main mix comprising from about 0.1% to about 1% of a carboxylic acid polymer thickening agent and water;
b. preparing a premix comprising silicone and from about 5% to about 35% sodium hyaluronate;
c. adding the premix to the main mix and milling the mixture to emulsify the silicone premix into the main mix to form an emulsion;
d. adding a polyacrylamide polymer and copolymer thickening agent to the emulsion to form a fully hydrated, physically stable skin care composition comprising an average viscosity greater than 25,000 cP.

12. The method of claim 11, wherein the method is performed without adding heat.

13. The method of claim 11, wherein the main mix further comprises glycerin.

14. The method of claim 11, wherein the premix is substantially free of water or free of water.

15. The method of claim 11, wherein the physically stable skin care composition is homogenous.

16. The method of claim 11, wherein the premix comprises from about 9% to about 25% sodium hyaluronate.

17. The method of claim 11, wherein the premix is added to the main mix in line at a transfer injection port.

18. The method of claim 17, wherein the amount of sodium hyaluronate in line at the transfer injection port when the premix and main mix first meet is less than 2%.

19. The method of claim 11, wherein one or more additional ingredients are added to the emulsion; wherein the additional ingredients are selected from vitamin B₃ compound, preservative, additional skin care active, pH adjuster, emollient, and combinations thereof.

20. The method of claim 19, wherein the additional ingredient comprises a vitamin B₃ compound selected from niacinamide, nicotinic acid, nicotinyl alcohol, and combinations thereof is added to the emulsion.
